(19) United States
(12) Patent Application Publication
(10) Pub. No.: US 2025/0092425 A1
(43) Pub. Date: Mar. 20, 2025

(54) FgTad2-FgTad3-Ame1 EDITING ENZYME SYSTEM, EDITING TOOL, AND EDITING METHOD

(71) Applicant: Northwest A&F University, Xianyang City (CN)

(72) Inventors: Huiquan LIU, Xianyang City (CN); Chanjing FENG, Xianyang City (CN); Yanfei DU, Xianyang City (CN); Kaiyun XIN, Xianyang City (CN); Jingwen ZOU, Xianyang (CN); Qinhu WANG, Xianyang City (CN); Cong JIANG, Xianyang City (CN)

(21) Appl. No.: 18/417,394

(22) Filed: Jan. 19, 2024

(30) Foreign Application Priority Data
Sep. 19, 2023 (CN) 2023112129760

(51) Int. Cl. C12N 15/90; C12N 9/22; C12N 9/78; C12N 15/11; C12N 15/85

(52) U.S. Cl. CPC C12N 15/90; C12N 9/22; C12N 9/78; C12N 15/11; C12N 15/85; C12Y 305/04; C12N 2310/20

(57) ABSTRACT

The present disclosure belongs to the technical field of gene editing, and specifically provides a FgTad2-FgTad3-Amel editing enzyme system, an editing tool, and an editing method. In the present disclosure, the system is a ternary complex composed of three genes (proteins), FgTAD2, FgTAD3, and AME1, and can catalyze A-to-I editing on mRNAs. Extensive A-to-I mRNA editing can be detected by expressing the AME1 gene in vegetative stage of Fusarium graminearum. Transforming the FgTad2-FgTad3-Ame1 editing enzyme system into Saccharomyces cerevisiae, Escherichia coli, and human cells to allow expression can produce A-to-I mRNA editing. This indicates that a Fusarium graminearum-derived A-to-I mRNA editing system is active and broadly adaptable, and has a great potential to be developed as a gene editing tool in different organisms.

### CROSS REFERENCE TO RELATED APPLICATION

This patent application claims the benefit and priority of Chinese Patent Application No. 2023112129760, filed with the China National Intellectual Property Administration on Sep. 19, 2023, the disclosure of which is incorporated by reference herein in its entirety as part of the present application.

### REFERENCE TO SEQUENCE LISTING

A computer readable XML file entitled “GWP20230604789”, created on Dec. 26, 2023, with a file size of about 59,475 bytes, contains the sequence listing for this application, has been filed with this application, and is hereby incorporated by reference in its entirety.

### TECHNICAL FIELD

The present disclosure belongs to the technical field of gene editing, and in particular relates to a FgTad2-FgTad3-Ame1 editing enzyme system, an editing tool, and an editing method.

### BACKGROUND

A-to-I mRNA editing is an important RNA modification that is prevalent in animals. Specifically, enzymes from an Adenosine Deaminase Acting on RNA (ADAR) family convert adenosine (A) into inosine (I) through deamination, and the inosine is recognized as guanosine (G) by various cellular processes. Therefore, A-to-I mRNA editing is equivalent to an A-to-G mutation in the mRNA, and it can result in alterations in the protein encoded by the mRNA. The applicant's team has previously discovered A-to-I mRNA editing in fungi such as Fusarium graminearum and Neurospora crassa, and showed that it occurred specifically during sexual reproduction^{[1][2]}. All ADARs share a common domain architecture consisting of a variable number of N-terminal dsRNA binding domains (dsRBDs) and a C-terminal catalytic deaminase domain^{[3]}. The existence of a diversified gene family in the earliest branching lineages of animals, but not in their close unicellular holozoan and fungal relatives, is consistent with this gene family being an animal-specific innovation^{[4]}. Therefore, the catalytic system of A-to-I mRNA editing in fungi is still unclear.

### CITED REFERENCES

[1] Liu H, Wang Q, He Y, Chen L, Hao C, Jiang C et al. 2016. Genome-wide A-to-I RNA editing in fungi independent of ADAR enzymes. Genome Res, 26 (4), 499-509.
[2] Liu H, Li Y, Chen D, Qi Z, Wang Q, Wang J, Jiang C, Xu J R. 2017 A-to-I RNA editing is developmentally regulated and generally adaptive for sexual reproduction in Neurospora crassa. Proc Natl Acad Sci USA, 114 (37), E7756-E7765.
[3] Savva Y A, Rieder L E, Reenan R A. 2012. The ADAR protein family. Genome Biol 13: 252.
[4] Grice L F., Degnan, B M. 2015. The origin of the ADAR gene family and animal RNA editing. BMC Evol Biol, 15(1), 4.

### SUMMARY

An objective of the present disclosure is to provide a FgTad2-FgTad3-Ame1 editing enzyme system derived from Fusarium graminearum, an editing tool, and an editing method. In the present disclosure, genes from the editing enzyme system in Fusarium graminearum can conduct specific A to I(G) editing on the mRNA of each species.

The present disclosure provides a FgTad2-FgTad3-Ame1 editing enzyme system, where Ame1 has an amino acid sequence shown in SEQ ID NO: 1, FgTad2 has an amino acid sequence shown in SEQ ID NO: 2, and FgTad3 has an amino acid sequence shown in SEQ ID NO: 3.

Preferably, a nucleotide sequence encoding the Ame1 is shown in SEQ ID NO: 4, a nucleotide sequence encoding the FgTad2 is shown in SEQ ID NO: 5, and a nucleotide sequence encoding the FgTad3 is shown in SEQ ID NO: 6.

The present disclosure further provides use of the editing enzyme system as an A-to-I mRNA editing tool.

The present disclosure further provides an A-to-I mRNA editing tool, including the editing enzyme system.

The present disclosure further provides a recombinant vector, including an encoding gene of the editing enzyme system.

Preferably, when the editing enzyme system is expressed in Saccharomyces cerevisiae, a pYES2 vector is used as a basic vector;
when the editing enzyme system is expressed in Escherichia coli, a pRSFDuet1 vector is used as the basic vector; and
when the editing enzyme system is expressed in a human cell, a pCMV-Blank vector is used as the basic vector.

The present disclosure further provides a construction method of the recombinant vector, including: inserting an AME1 gene expression cassette, an FgTAD2 gene expression cassette, and an FgTAD3 gene expression cassette into the basic vector.

The present disclosure further provides a method for A-to-I mRNA editing using the A-to-I mRNA editing tool, including: transforming the editing enzyme system, or the A-to-I mRNA editing tool, or the recombinant vector into a species that requires the A-to-I mRNA editing.

Preferably, the species includes a prokaryote and a eukaryote.

Preferably, the species includes Saccharomyces cerevisiae, the Escherichia coli, and the human cells.

Beneficial effects: this disclosure presents a ternary complex enzyme system for editing, called FgTad2-FgTad3-Ame1. The system comprises three genes (proteins): FgTAD2, FgTAD3, and AME1. A heterodimer formed by FgTad2 and FgTad3 can perform A-to-I editing at position 34 of the tRNA anticodon loops. On the other hand, Ame1 interacts with the N-terminal domain of FgTad3, which leads to the formation of a FgTad2-FgTad3-Ame1 ternary complex that can catalyze A-to-I editing on mRNA. While FgTAD2 and FgTAD3 genes are expressed constitutively, the AME1 gene is expressed specifically during the sexual stage, and A-to-I mRNA editing only occurs during this stage under natural conditions. By expressing the AME1 gene in the vegetative stage of Fusarium graminearum, extensive A-to-I mRNA editing can be detected. Furthermore, transferring the FgTad2-FgTad3-Ame1 editing enzyme system to Saccharomyces cerevisiae, Escherichia coli, and human cell lines for expression can result in A-to-I mRNA editing. This implies that a Fusarium graminearum-derived A-to-I mRNA editing system is active and broadly adaptable, with great potential to be developed as a gene editing tool in different organisms.

### BRIEF DESCRIPTION OF THE DRAWINGS

FIG. 1 shows a diagram illustrating an evolutionary relationship of AME1, an activator of mRNA editing in the present disclosure, in the class Sordariomycetes; where the AME1 is a branch specifically evolved in the class Sordariomycetes, and participates in mRNA editing to play a role in activating the mRNA editing;

FIG. 2 shows a schematic diagram for constructing a S. cerevisiae expression vector pYES2-Ame1 of a FgTad2-FgTad3-Ame1 editing enzyme system in an example of the present disclosure;

FIG. 3 shows a schematic diagram for constructing a S. cerevisiae expression vector pYES2-Leu-4M-FgTad2 of a FgTad2-FgTad3-Ame1 editing enzyme system in an example of the present disclosure;

FIG. 4 shows a schematic diagram for constructing a S. cerevisiae expression vector pYES2-Trp-FgTad3 of a FgTad2-FgTad3-Ame1 editing enzyme system in an example of the present disclosure;

FIGS. 5A-D show peak diagrams of sites where editing occurs on a substrate mRNA when the FgTad2-FgTad3-Ame1 editing enzyme system is transformed into S. cerevisiae protoplast in an example of the present disclosure;

FIGS. 6A-B show mRNA editing events occurring in the S. cerevisiae and E. coli and editing levels thereof after the FgTad2-FgTad3-Ame1 editing enzyme system is transferred into the E. coli protoplast and S. cerevisiae protoplast and their transcriptomes are sequenced in an example of the present disclosure; where the S. cerevisiae and E. coli are shown from left to right in sequence;

FIGS. 7A-C show comparison results of a sequence preference of mRNA editing and a sequence preference of mRNA editing in F. graminearum after the FgTad2-FgTad3-Ame1 editing enzyme system is transferred into the E. coli protoplast and S. cerevisiae protoplast and a transcriptome is sequenced in an example of the present disclosure; where the F. graminearum, E. coli, and S. cerevisiae are shown from top to bottom in sequence;

FIG. 8 shows a schematic diagram for constructing an E. coli expression vector of the FgTad2-FgTad3-Ame1 editing enzyme system in an example of the present disclosure;

FIGS. 9A-B show peak diagrams of sites where editing occurs on a substrate mRNA when the FgTad2-FgTad3-Ame1 editing enzyme system is transformed into the E. coli in an example of the present disclosure;

FIG. 10 shows a schematic diagram for constructing a human cell expression vector of the FgTad2-FgTad3-Ame1 editing enzyme system in an example of the present disclosure; and

FIGS. 11A-B show peak diagrams of sites where editing occurs on the AME1 gene itself when the FgTad2-FgTad3-Ame1 editing enzyme system is transferred into a human pancreatic cancer cell in an example of the present disclosure.

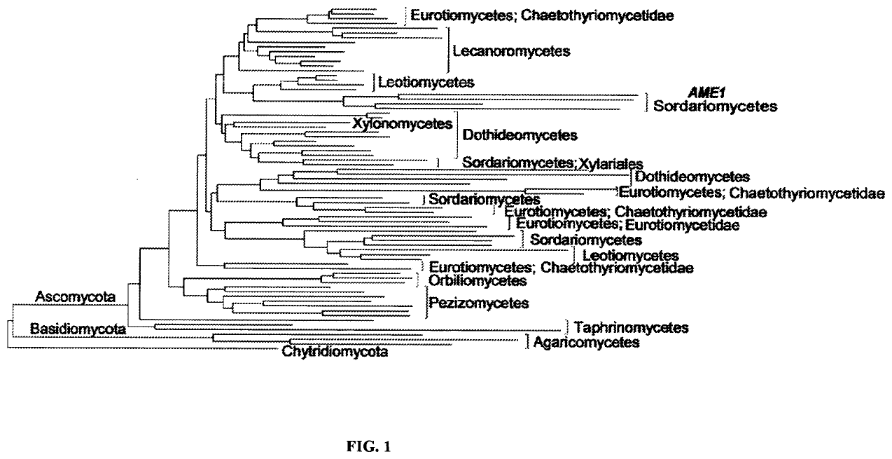

### DETAILED DESCRIPTION OF THE EMBODIMENTS

The present disclosure provides a FgTad2-FgTad3-Ame1 editing enzyme system, where Ame1 has an amino acid sequence shown in SEQ ID NO: 1, FgTad2 has an amino acid sequence shown in SEQ ID NO: 2, and FgTad3 has an amino acid sequence shown in SEQ ID NO: 3.

In the present disclosure, the FgTad2-FgTad3-Ame1 editing system in E graminearum is taken as an example. However, according to the present disclosure, the system in other fungi of the class Sordariomycetes should also have the function of mRNA editing, and therefore should also fall within the protection scope of the present disclosure.

In the present disclosure, the Ame1 is preferably derived from F. graminerum, and has an amino acid sequence shown in SEQ ID NO: 1, with a total of 652 amino acids. A corresponding AME1 gene has a nucleotide sequence shown in SEQ ID NO: 4, with a sequence length of 1,959 bp.

In the present disclosure, the FgTad2 (denoted as FgTad2-4M in the examples) is preferably derived from F. graminerum, and has an amino acid sequence shown in SEQ ID NO: 2, with a total of 377 amino acids. A corresponding FgTAD2 gene has a nucleotide sequence shown in SEQ ID NO: 5, with a sequence length of 1,134 bp.

In the present disclosure, the FgTad3 is preferably derived from F. graminerum, and has an amino acid sequence shown in SEQ ID NO: 3, with a total of 428 amino acids. A corresponding FgTAD3 gene has a nucleotide sequence shown in SEQ ID NO: 6, with a sequence length of 1,287 bp.

In the present disclosure, the AME1 gene has a nucleotide sequence shown in SEQ ID NO: 7 after codon optimization in E. coli, with a full length of 1,959 bp.

In the present disclosure, the FgTAD2 gene has a nucleotide sequence shown in SEQ ID NO: 8 after codon optimization in E. coli, with a full length of 1,134 bp.

In the present disclosure, the FgTAD3 gene has a nucleotide sequence shown in SEQ ID NO: 9 after codon optimization in E. coli, with a full length of 1,287 bp.

The present disclosure further provides use of the editing enzyme system as an A-to-I mRNA editing tool.

The present disclosure provides an example in which the A-to-I mRNA editing catalytic system in F. graminearum is a FgTad2-FgTad3-Ame1 ternary complex. Through various experiments, including gene knockout, overexpression, co-IP, RNA-seq, RIP-seq, and heterologous expression, it has been demonstrated that the FgTad2-FgTad3 enzyme responsible for A-to-I editing of position A34 on the tRNA anticodon loops is also an enzyme that catalyzes A-to-I mRNA editing. However, the ability of FgTad2-FgTad3 to edit mRNA depends on the presence of the Ame1 protein. The AME1 gene is specifically induced and expressed during the sexual reproduction of F. graminearum, resulting in natural A-to-I mRNA editing only occurring during this stage. Extensive A-to-I mRNA editing can be detected by expressing the AME1 gene in the vegetative stage of F. graminearum. The C-terminal of FgTad3 interacts with FgTad2, while the N-terminal of FgTad3 interacts with Ame1, forming the FgTad2-FgTad3-Ame1 ternary complex responsible for A-to-I mRNA editing. Transferring the FgTad2-FgTad3-Ame1 editing enzyme system into S. cerevisiae, human cell lines, and E. coli can result in A-to-I mRNA editing, indicating that this system is active and broadly adaptable, with great potential as a gene editing tool in different organisms. Furthermore, it has been found that the function of Ame1 in mRNA editing is conserved among fungi of the class Sordariomycetes. The orthologous genes of AME1 are found to be widely distributed among ascomycetes. Furthermore, an ancestral gene replication event took place in the last common ancestor of fungi from the class Sordariomycetes and its closest related class, Leotiomycetes. The AME1 gene has an accelerated evolution rate in fungi from the class Sordariomycetes compared to those from the class Leotiomycetes. The AME1 ortholog of Sclerotinia sclerotiorum cannot replace the function of the AME1 gene in F. graminearum, indicating that the function of the AME1 gene in mRNA editing has specifically evolved from fungi of the class Sordariomycetes. Since A-to-I mRNA editing is prevalent in fungi of the class Sordariomycetes, it is expected that the AME1 gene in these fungi generally has the function of mRNA editing. Therefore, the present disclosure demonstrates that the A-to-I mRNA editing catalytic system in F. graminearum has great potential as a broad-spectrum gene editing tool.

The present disclosure further provides an A-to-I mRNA editing tool, including the editing enzyme system.

The present disclosure further provides a recombinant vector, including an encoding gene of the editing enzyme system.

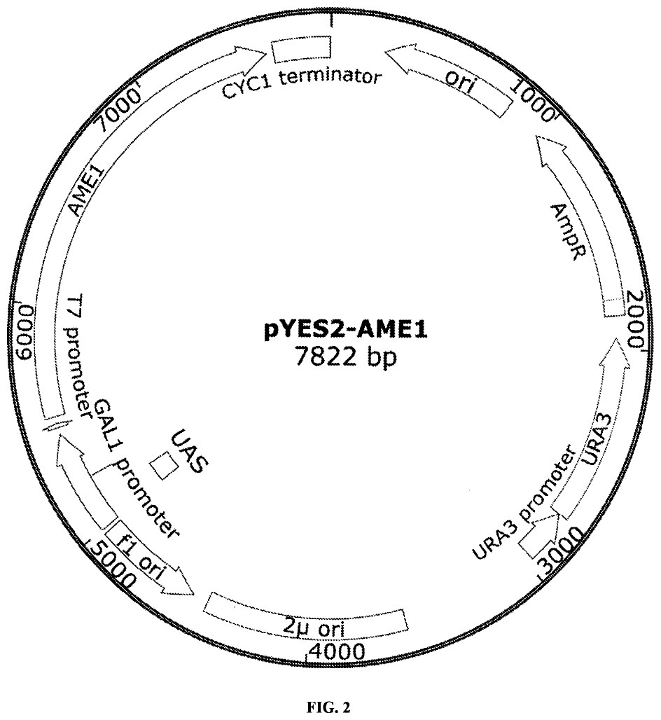

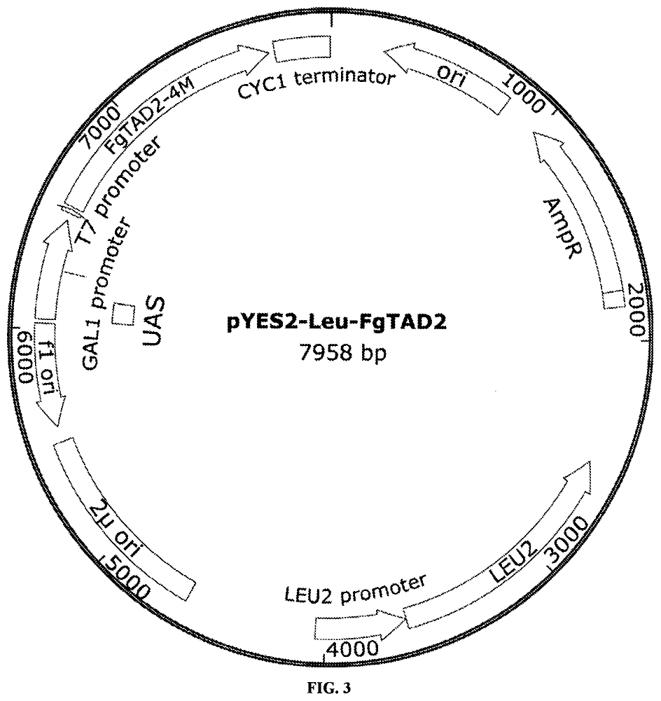

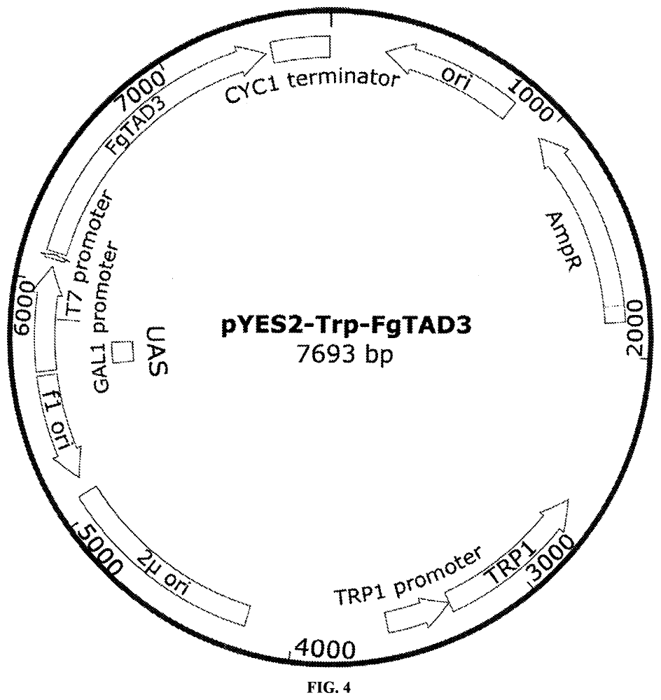

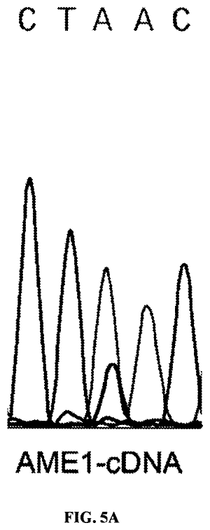

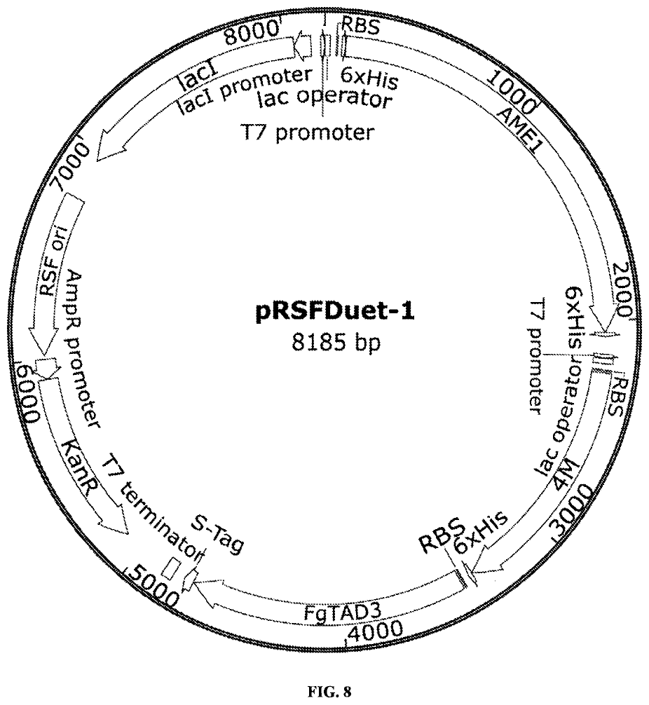

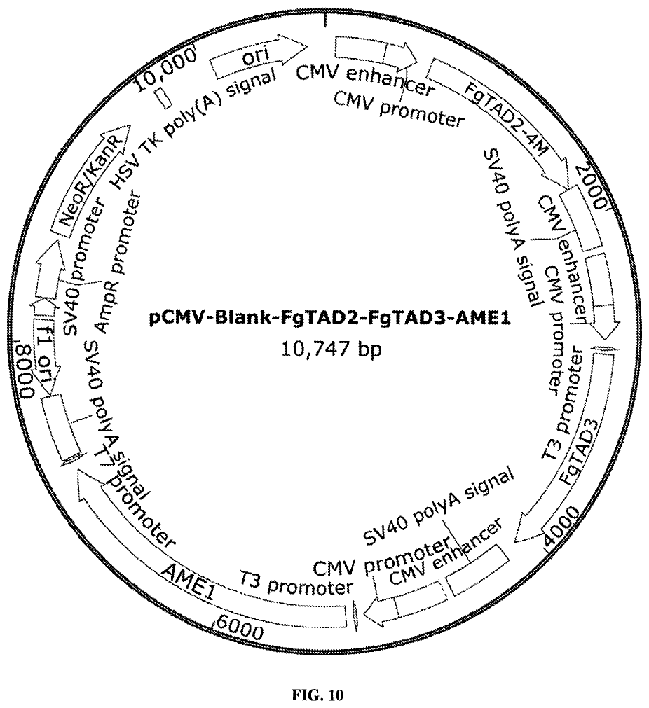

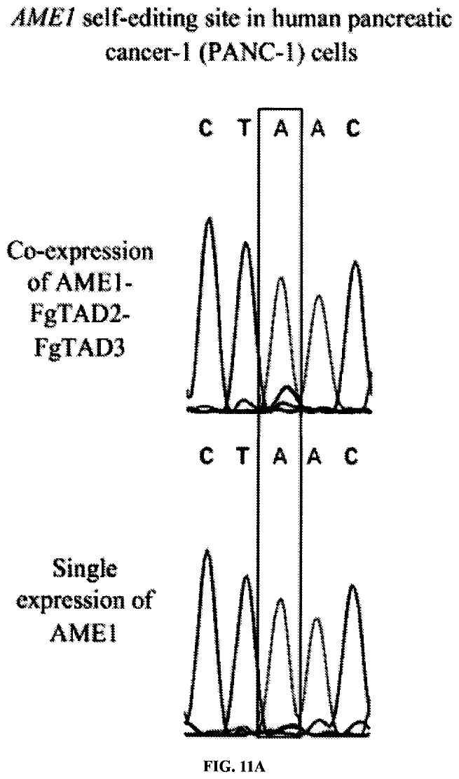

In the present disclosure, the pYES2, pRSFDuet1, and pCMV-Blank vectors are preferably used as basic vectors for expressing the editing enzyme system in S. cerevisiae, E. coli, and human cells, respectively. Moreover, after adding corresponding promoter and terminator to each of the basic vectors, resulting novel vectors are transformed into multiple expression systems, and the constructed recombinant vectors have plasmid maps shown in preferably FIG. 2, FIG. 3, FIG. 4, FIG. 8, and FIG. 10.

The present disclosure further provides a construction method of the recombinant vector, including: inserting an AME1 gene expression cassette, an FgTAD2 gene expression cassette, and an FgTAD3 gene expression cassette into the basic vector.

In the present disclosure, the gene expression cassette includes preferably a promoter, a gene, and a terminator, and the promoter and the terminator used in different species are different to allow efficient expression. For example, when being expressed in S. cerevisiae, the AME1, FgTAD2, and FgTAD3 genes are expressed using a promoter GAL1 and a terminator CYC1 for gene expression on the pYES2 vector. When being expressed in E. coli, the expression cassettes of the AME1, FgTAD2, and FgTAD3 genes are inserted into the pRSFDuet1 vector, and are respective codon-optimized encoding region sequences, and include a T7 promoter and a T7 terminator. For example, when being expressed in human cells, the expression cassettes of the AME1, FgTAD2, and FgTAD3 genes are inserted into the pCMV-Blank vector, and are the respective encoding region sequences, and include a CMV promoter and an SV40 polyadenylation signal.

In the present disclosure, there is no special limitation on a construction method of the recombinant vector, and conventional construction methods can be used. The constructed recombinant vector has nucleotide sequence maps preferably shown in FIG. 2, FIG. 3, FIG. 4, FIG. 8, and FIG. 10.

The present disclosure further provides a method for A-to-I mRNA editing using the A-to-I mRNA editing tool, including: transforming the editing enzyme system, or the A-to-I mRNA editing tool, or the recombinant vector into a species that requires the A-to-I mRNA editing.

In the present disclosure, the species includes preferably a prokaryote and a eukaryote. In the examples, S. cerevisiae, E. coli, and human cells are used as examples for illustration, but they cannot be regarded as the entire protection scope of the present disclosure.

To further illustrate the present disclosure, the FgTad2-FgTad3-Ame1 editing enzyme system, the editing tool, and the editing method provided by the present disclosure are described in detail below in connection with examples, but these examples should not be construed as limiting the claimed scope of the present disclosure.

In the examples of the present disclosure, the operations used are routine experimental operations in the art:

An amplification system for PCR amplification is 200 μL, including: 20 μL of 10×Taq Reaction Buffer, 16 μL of dNTP (2.5 mM), 4 μL of Forward Primer (2 mM), 4 μL of Reverse Primer (2 mM) (Tables 1 to 3), 4 μL of a template (genomic DNA or cDNA), 2 μL of ExTaq polymerase (5 U/μL), and ddH₂O as a balance.

TABLE 1
Primers and sequences for construction of
recombinant vector on pYES2

| | Primer | SEQ ID |
|---|---|---|
| Primer name | sequence | NO: |
| AME1-pYES2-F | TCACTATAGG | 10 |
| | GAATATTAAG | |
| | CTTCTACGCA | |
| | AAAACGGCAT | |
| | CATG | |
| AME1-pYES2-R | CCCTCTAGAT | 11 |
| | GCATGCTCGA | |
| | GCTAAGCGTT | |
| | AAGGTCCAGG | |
| | TCC | |
| pYES2-FgTAD2-4M-F | TCACTATAGG | 12 |
| | GAATATTAAG | |
| | CATGCAATCT | |
| | CTCCAGAATC | |
| | GACT | |
| pYES2-FgTAD2-4M-R | CCCTCTAGAT | 13 |
| | GCATGCTCGA | |
| | GCTAGACATT | |
| | GGTGCGATCT | |
| | GCCAG | |
| pYES2-FgTAD3-F | TCACTATAGG | 14 |
| | GAATATTAAG | |
| | CATGTCCACC | |
| | GAACTGGCTC | |
| pYES2-FgTAD3-R | CCCTCTAGAT | 15 |
| | GCATGCTCGA | |
| | GCTAAACATG | |
| | AGTGATGGGA | |
| | TCAAGAG | |
| pYES2-Leu2-F | GAATTAATTC | 16 |
| | TCATCTTTGA | |
| | CAGCTAACTG | |
| | TGGGAATACT | |
| | CAGGTATCG | |
| pYES2-Leu2-R | ATAATGCCTT | 17 |
| | TAGCGGCTTA | |
| | ACTGTCCGGT | |
| | CGAAATTCCC | |
| | CTACC | |
| pYES2-TRP1-F | GAATTAATTC | 18 |
| | TCATCTTTGA | |
| | CAGCTTTGTA | |
| | CTGAGAGTGC | |
| | ACCATAGATC | |
| pYES2-TRP1-R | ATAATGCCTT | 19 |
| | TAGCGGCTTA | |
| | ACTGTCCTGA | |
| | TGCGGTATTT | |
| | TCTCCTT | |

TABLE 2
Primers and sequences for construction of
recombinant vector on pRSFDuet1

| | Primer | SEQ ID |
|---|---|---|
| Primer name | sequence | NO: |
| PRSFDuet-AME-MCS1-F | AGCAGCCATC | 20 |
| | ACCATCATCA | |
| | CCACATGACC | |
| | CGCGGCGGCC | |
| | GCA | |
| PRSFDuet-AME-MCS1-R | TTATGCGGCC | 21 |
| | GCAAGCTTGT | |
| | CGACTTAGTG | |
| | GTGGTGGTGG | |
| | TGGTGCG | |
| PRSFDuet-4M-MCS2-F | TTAAGTATAA | 22 |
| | GAAGGAGATA | |
| | TACATATGCA | |
| | GAGCCTGCAG | |
| | AACCG | |
| PRSFDuet-4M-MCS2-R2 | TACCGACGTC | 23 |
| | AGCGATCGCG | |
| | TGGCCGGCCT | |
| | TAGTGGTGGT | |
| | GGTGGTGGTG | |
| | CAC | |
| PRSFDuet-T3-MCS2-F | CCGGCCACGC | 24 |
| | GATCGCTGAC | |
| | GTCAAGGAGA | |
| | TATACATATG | |
| | AGCACCGAAC | |
| | TGGCGCC | |
| PRSFDuet-T3-MCS2-R | CAAATTTCGC | 25 |
| | AGCAGCGGTT | |
| | TCTTTTTACA | |
| | CGTGGGTAAT | |
| | CGGATCCAGC | |
| PRS-MCS1-F | GGATCTCGAC | 26 |
| | GCTCTCCCT | |
| PRS-MCS1-R | GATTATGCGG | 27 |
| | CCGTGTACAA | |
| PRS-MCS2-F | TTGTACACGG | 28 |
| | CCGCATAATC | |
| PRS-MCS2-R | GCTAGTTATT | 29 |
| | GCTCAGCGG | |

TABLE 3
Primers and sequences for construction of
recombinant vector on pCMV-Blank

| | Primer | SEQ ID |
|---|---|---|
| Primer name | sequence | NO: |
| pCMV-AME1-F | ACTAAAGGGA | 30 |
| | ACAAAAGCTG | |
| | GAGCTCCACC | |
| | CTACGCAAAA | |
| | ACGGCATCAT | |
| | G | |
| AME1-SV40-R | CAGGCTGCGC | 31 |
| | AACTGTTGGG | |
| | AAGGGCGACT | |
| | AAGCGTTAAG | |
| | GTCCAGGTCC | |
| SV40-F | TCGCCCTTCC | 32 |
| | CAACAGTTG | |
| Sv40-R | TTAAGATACA | 33 |
| | TTGATGAGTT | |
| | TGGACAAACC | |
| | AC | |
| proCMV-SV40-F | GTGGTTTGTC | 34 |
| | CAAACTCATC | |
| | AATGTATCTT | |
| | AATCAATTAC | |
| | GGGGTCATTA | |
| | GTTCATAGC | |
| proCMV-R | GGTGGAGCTC | 35 |
| | CAGCTTTTGT | |
| proCMV-4M-F | ACTAAAGGGA | 36 |
| | ACAAAAGCTG | |
| | GAGCTCCACC | |
| | ATGCAATCTC | |
| | TCCAGAATCG | |
| | ACT | |
| 4M-SV40-R | TCAGGCTGCG | 37 |
| | CAACTGTTGG | |
| | GAAGGGCGAC | |
| | TAGACATTGG | |
| | TGCGATCTGC | |
| proCMV-FgTAD3-F | ACTAAAGGGA | 38 |
| | ACAAAAGCTG | |
| | GAGCTCCACC | |
| | ATGTCCACCG | |
| | AACTGGCTC | |
| FgTAD3-SV40-R | TTCTAGACTC | 39 |
| | GAGAGATCTG | |
| | TCGACGATAT | |
| | CTCAACTCCG | |
| | GAGCTGACAT | |

An amplification program includes: initial denaturation at 94° C. for 3 min; denaturation at 94° C. for 40 sec, annealing at 56° C. to 58° C. for 30 sec, extension at 72° C. for 2 min, conducting 34 cycles; extension at 72° C. for 10 min; and 16° C., forever.

Recovery, ligation, and cloning of DNA fragments: the fragments separated by electrophoresis after amplification are recovered using an agarose gel DNA recovery kit (Guangzhou Magen Biotechnology Co., Ltd), and detailed steps refer to instructions.

The recovered product is ligated to the corresponding vector: ligation system and reaction conditions: 1 μg of the product recovered by PCR and 100 ng of the vector; reaction at 50° C. for 15 min. A resulting ligation product is transformed into E. coli DH5α. After resistance screening, single clones are selected and sent to Sangon Biotech (Shanghai) Co., Ltd. to allow sequencing.

S. cerevisiae transformation: the vector with correct sequencing is transferred into a S. cerevisiae INVSc1 strain. The constructed editing vector and editing reporter vector are co-transfected into INVSc1 competent cells using a heat-shock transformation method, and then screened using a nutritional deficiency medium. The strains obtained after screening are tested by colony PCR to ensure the success co-transformation of the vectors.

RNA extraction: the RNA extraction is conducted using a Promega RNA extraction kit (Promega Biotechnology Co., Ltd., Beijing), and the detailed steps are referred to the instructions.

RNA reverse transcription: the RNA reverse transcription is conducted using a TIANGEN one-step reverse transcription PCR kit (TIANGEN Biotech (Beijing) Co., Ltd), and the detailed steps are referred to the instructions.

RNA-seq sequencing: the S. cerevisiae samples transformed the vector are collected and sent to Novogene (Novogene Technology Co., Ltd., Beijing) to allow the RNA-seq sequencing.

### Example 1

Construction of Recombinant Vector on pYES2

PCR amplification primers of 5 fragments were designed together with at least homologous sequences of their upstream or downstream fragments (Table 1), where the 5 fragments included an Ame1 encoding sequence (SEQ ID NO: 4), a FgTad2-4M encoding region sequence (SEQ ID NO: 5), an FgTad3 encoding sequence (SEQ ID NO: 6), Leu2 (amplified from pGADT7 plasmid), and Trp1 (amplified from pGBKT7 plasmid).

PCR amplification was conducted by a conventional method using the above primers to recover DNA fragments. The pYES2 vector was double-digested using NcoI and ClaI restriction sites, and the Leu2 and Trp1 were amplified and replaced with a Ura3 gene through one-step cloning to create pYES2-Leu and pYES2-Trp vectors. The Ame1, FgTad2-4M, and FgTad3 were constructed into the pYES2, pYES2-Leu, and pYES2-Trp vectors through one-step cloning using the BamHI and NotI restriction sites, respectively. The one-step cloning system was as follows: vector: 100 ng; DNA: 1 μg; NovoRec® 10× reaction buffer: 2 μL; NovoRec® recombinase: 1 μL; making up to 20 μL with sterile water; reaction at 50° C. for 15 min.

10 μL of a resulting ligation product was added to DH5α to allow heat-shock transformation. The reaction steps were as follows: ice bath for 30 min; heat shock at 42° C. for 90 sec; ice bath for 5 min; 500 μL sterile LB was added to allow shaking culture in a 37° C. incubator for 1 h; the bacteria were collected by centrifugation and resuspended with sterile water; the bacterial solution was then spread on a 90 mm 10 mL LB solid plate; the cells were cultured at 37° C. for 10 h to 12 h; single colonies that grew out were tested by PCR and sent to Shanghai Sangon for sequencing. A successfully constructed vector was shown in FIG. 2, FIG. 3 and FIG. 4.

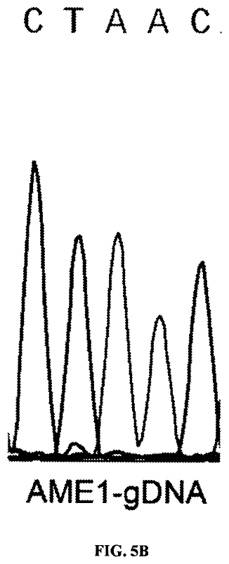

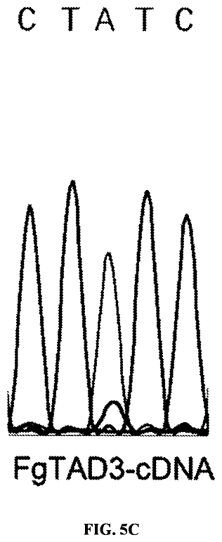

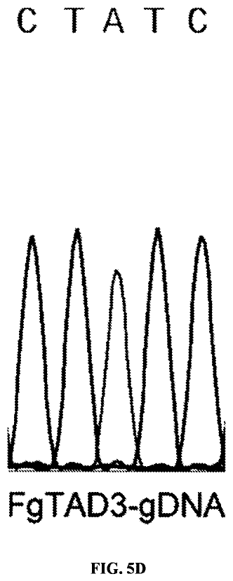

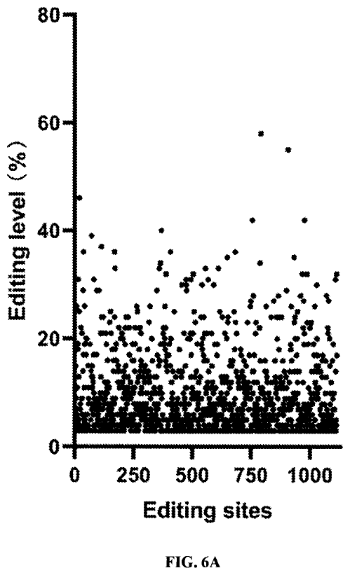

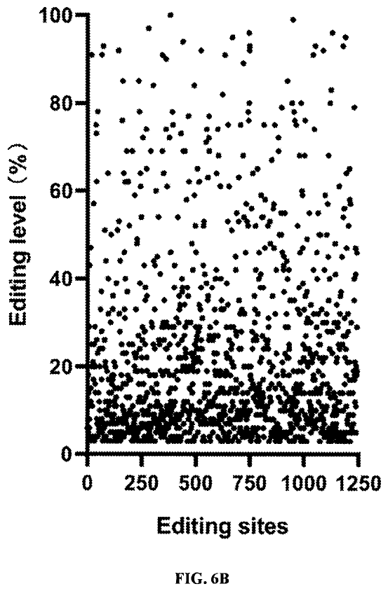

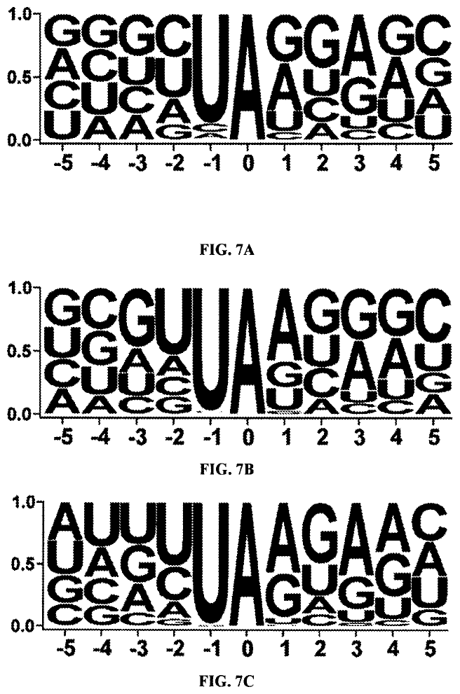

The above vectors constructed from pYES2-Ame1, pYES2-Leu-4M-FgTad2, and pYES2-Trp-FgTad3 vector plasmids using homologous recombination were used as editing vectors in S. cerevisiae. The pYES2 editing vector was transformed into INVSc1 competent cells by heat-shock transformation. A single colony that had been successfully transformed was cultured with shaking in 10 mL of 2% glucose SD-Ura-Leu-Trp liquid medium at 30° C. for 24 h, centrifuged and a supernatant medium was discarded, 10 mL of 2% galactose SD-Ura-Leu-Trp liquid medium was added to allow culture with shaking at 30° C. for 12 h, and centrifuged to collect samples, gDNA and RNA were then extracted, and the RNA underwent reverse transcription to obtain cDNA. Since AME1 and FgTAD3 had their own editing sites, cDNA and gDNA were used as templates to amplify an editing substrate, and Sanger sequencing was conducted to determine whether the editing substrate was edited at the RNA level (FIGS. 5A-D) and gDNA sequencing results were used as a control. RNA-seq and DNA-seq high-throughput sequencing were conducted on the edited S. cerevisiae strains identified by Sanger sequencing, and RNA editing events that occurred in the S. cerevisiae transcriptome were identified through bioinformatics analysis (FIG. 6A). The 1,144 editing sites in S. cerevisiae were identified, and the analysis found that the editing substrate had a sequence preference consistent with that in F. graminearum (FIG. 7C). This indicated that the mRNA editing complex from F. graminearum played the role of mRNA editing.

### Example 2

Construction of Recombinant Vector on pRSFDuet1

PCR amplification primers of 3 fragments were designed together with homologous sequences of their upstream or downstream fragments (Table 2), where the three primers included optimized Ame1 encoding sequence (SEQ ID NO: 7) that was easy to express in E. coli, optimized FgTad2-4M encoding region sequence (SEQ ID NO: 8), and optimized FgTad3 encoding sequence (SEQ ID NO: 9);

PCR amplification was conducted by a conventional method using the above primers to recover DNA fragments, a pRSFDuet1 plasmid vector was double-digested with BamHI and EcoRI to linearization. An amplified Ame1 fragment was ligated to an enzyme-digested vector through one-step cloning; the vector ligated to the Ame1 encoding sequence was double-digested with KpnI and XhoI; the FgTad3 encoding sequence fragment was ligated to a double-digested vector through one-step cloning. In the same way, a ligated vector was double-digested with BgIII and EcoRV, and then ligated to the FgTad2-4M encoding region sequence. A ligation system and reaction steps were the same as those in Example 1. The pRSFDuet1 plasmid included a T7 promoter (with nucleotide sequence shown in SEQ ID NO: 40), a lac operator (with nucleotide sequence shown in SEQ ID NO: 41), and a T7 terminator (with nucleotide sequence shown in SEQ ID NO: 42).

10 μL of the ligation product was added to DH5α for heat-shock transformation, and the reaction steps were the same as those in Example 1; the grown single colonies were detected by PCR and sent to Shanghai Sangon to allow sequencing, and finally a vector was successfully constructed as shown in FIG. 8.

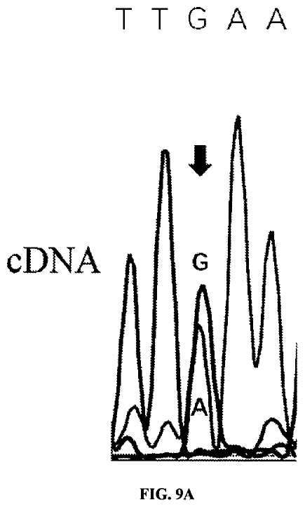

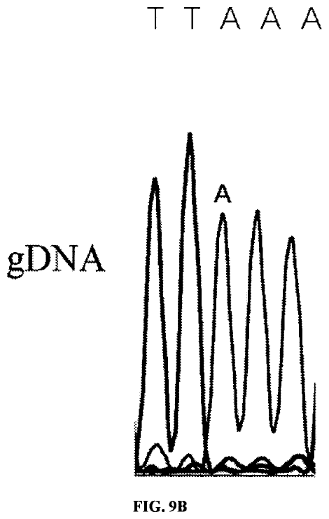

The fragments recovered from the CDS regions of AME1, FgTAD2, and FgTAD3 were constructed into the pRSFDuet1 plasmid using homologous recombination to obtain an E. coli editing vector. The pRSFDuet1 editing vector was transformed into E. coli BL21 competent cells by heat-shock transformation. A successfully transformed E. coli colony was subjected to shaking culture, gDNA and RNA were then extracted, RNA underwent reverse transcription to obtain cDNA. Since FgTAD3 had its own editing sites, cDNA and gDNA were used as templates to amplify an editing substrate, and Sanger sequencing was conducted to determine whether the editing substrate was edited at the RNA level (FIGS. 9A-B) and the gDNA sequencing results were used as a control. Sanger sequencing was conducted to identify the presence of edited E. coli, RNA-seq and DNA-seq high-throughput sequencing were conducted, and the RNA editing events occurring in E. coli were identified through bioinformatics analysis (FIG. 6B). The 1,241 editing sites were identified in E. coli, and the analysis found that the editing substrate sequence preference was consistent with that in F. graminearum (FIG. 7B), indicating that the mRNA editing complex in F. graminearum exerted the function of mRNA editing.

### Example 3

Construction of Recombinant Vector on pCMV-Blank

PCR amplification primers of 5 fragments were designed together with at least homologous sequences of their upstream or downstream fragments (Table 3), where the 5 fragments included an Ame1 encoding sequence, a CMV enhancer and promoter sequence (SEQ ID NO: 43), a SV40 polyA signal sequence (SEQ ID NO: 44), a FgTad2-4M encoding region sequence (SEQ ID NO: 5), and a FgTad3 encoding sequence.

After recovering DNA fragments, the fragments were then overlapped in vitro by PCR amplification according to the above sequences.

A pCMV-Blank plasmid vector was digested with HindIII to allow linearization, and the overlapped fragments were ligated to the digested vector through one-step cloning. The ligation product was added to DH5α for transformation, and ligation and transformation methods and reaction systems were the same as those in Example 1; the grown single colonies were detected by PCR and sent to Shanghai Sangon to allow sequencing, and finally a vector was successfully constructed as shown in FIG. 10.

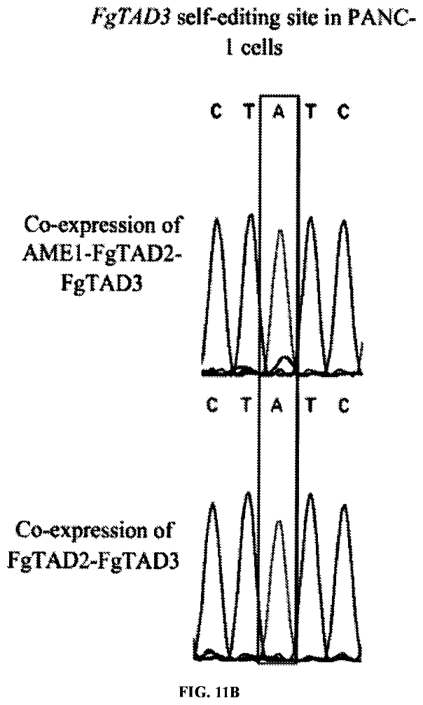

7×10⁵ pancreatic cancer cell lines PACN1 were plated in a 60 mm cell culture dish, cultured at 37° C. for 24 h, and the cells were then transfected with the constructed pCMV-Blank plasmid. A transfection reagent (TSnanofect from Beijing Tsingke Biotech Co., Ltd.) was gently shaken, and 5 μL of the transfection reagent was mixed with 120 μL of RNase-free water to reach 125 μL (on ice); while 125 μL of water was added to dilute 5 μg plasmid, and mixed gently. 125 μL of a diluted plasmid was added into 125 μL of a transfection reagent dilute solution to make a total volume of 250 μL, mixed gently, and incubated at room temperature for 15 min; 250 μL of a transfection solution was added to a cell culture dish, mixed gently, incubated at 37° C. for 48 h, and RNA was extracted; the RNA was reverse-transcribed to obtain cDNA. Since AME1 and FgTAD3 had their own editing sites, cDNA was used as a template to amplify the editing substrates, and Sanger sequencing was conducted to determine whether the editing substrates were edited (FIGS. 11A-B) and the cDNA templates with single transfection of AME1 or FgTAD2 and FgTAD3 were used as a negative control.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1-10. (canceled)

11. A FgTad2-FgTad3-Ame1 editing enzyme system, wherein Ame1 has an amino acid sequence shown in SEQ ID NO: 1, FgTad2 has an amino acid sequence shown in SEQ ID NO: 2, and FgTad3 has an amino acid sequence shown in SEQ ID NO: 3.

12. The editing enzyme system according to claim 11, wherein a nucleotide sequence encoding the Ame1 is shown in SEQ ID NO: 4, a nucleotide sequence encoding the FgTad2 is shown in SEQ ID NO: 5, and a nucleotide sequence encoding the FgTad3 is shown in SEQ ID NO: 6.

13. An A-to-I mRNA editing tool, comprising the editing enzyme system according to claim 11.

14. An A-to-I mRNA editing tool, comprising the editing enzyme system according to claim 12.

15. A recombinant vector, comprising an encoding gene of the editing enzyme system according to claim 11.

16. A recombinant vector, comprising an encoding gene of the editing enzyme system according to claim 12.

17. The recombinant vector according to claim 15, wherein when the editing enzyme system is expressed in S. cerevisiae, a pYES2 vector is used as a basic vector;
when the editing enzyme system is expressed in Escherichia coli, a pRSFDuet1 vector is used as the basic vector; and
when the editing enzyme system is expressed in a human cell, a pCMV-Blank vector is used as the basic vector.

18. The recombinant vector according to claim 16, wherein when the editing enzyme system is expressed in S. cerevisiae, a pYES2 vector is used as a basic vector;
when the editing enzyme system is expressed in Escherichia coli, a pRSFDuet1 vector is used as the basic vector; and
when the editing enzyme system is expressed in a human cell, a pCMV-Blank vector is used as the basic vector.

19. A construction method of the recombinant vector according to claim 15, comprising: inserting an AME1 gene expression cassette, an FgTAD2 gene expression cassette, and an FgTAD3 gene expression cassette into the basic vector.

20. A construction method of the recombinant vector according to claim 16, comprising: inserting an AME1 gene expression cassette, an FgTAD2 gene expression cassette, and an FgTAD3 gene expression cassette into the basic vector.

21. A construction method of the recombinant vector according to claim 17, comprising: inserting an AME1 gene expression cassette, an FgTAD2 gene expression cassette, and an FgTAD3 gene expression cassette into the basic vector.

22. A construction method of the recombinant vector according to claim 18, comprising: inserting an AME1 gene expression cassette, an FgTAD2 gene expression cassette, and an FgTAD3 gene expression cassette into the basic vector.

23. A method for A-to-I mRNA editing using the A-to-I mRNA editing tool according to claim 13, comprising: transforming the editing enzyme system into a species that requires A-to-I mRNA editing.

24. A method for A-to-I mRNA editing using the A-to-I mRNA editing tool according to claim 13, comprising: transforming the A-to-I mRNA editing tool into a species that requires A-to-I mRNA editing.

25. A method for A-to-I mRNA editing, comprising: transforming the recombinant vector according to claim 15 into a species that requires A-to-I mRNA editing.

26. The method according to claim 23, wherein the species comprises a prokaryote and an eukaryote.

27. The method according to claim 24, wherein the species comprises a prokaryote and an eukaryote.

28. The method according to claim 25, wherein the species comprises a prokaryote and an eukaryote.

29. The method according to claim 23, wherein the species comprises Saccharomyces cerevisiae, the Escherichia coli, and the human cell.

30. The method according to claim 26, wherein the species comprises Saccharomyces cerevisiae, the Escherichia coli, and the human cell.


SEQUENCE LISTING (44 sequences)

SEQ ID NO: 1 - protein, 652 aa, synthetic construct
  source 1..652: note = Amino acid sequence of Ame1
MTRGGRTSSVPRRPVDLTSNLSIAEKNDLTTLITAITEKLHNDISIIFDSPPVTPTLGDFSDIENVDIGHHHWLALSLQTDGKENVDPRHSNLSKNIPKPVPTDKITDQSRGSEGSEAITPQLRELKKEALIFFRKWQSIFLQRLRDISVTEPNGSQPNMRGRGRGRGSARGARGGRVARGGRGEGRGGLTLATGPPKTPSTHIDRELSRRFPPIPTSLWTLPLEKRKLLLHISLLIVISLQEYLAYARMTLLNLTSSLNLPLKVLHEEEKRIAQGLSQLAVDSVIEQASQQSQDENKPVLKSKARIAAMTGPFRLGTQLIAVGIGTAHGGHGLRLPTAAGLLGPMADNGPSTGSMLGIYSARPTCKMIESFSREIQDFGLMPLRCESQGDYMDATSIPSTARRLRLVIAINGWVTDKDNLTGSWKILGDNAEVYILRWEMNVLSNIGTALETVIRSSAWTAAKQAISSRTIFKNLIEATWPMELMKVSKIIDNPWSMGMVRAEKAGAVLADAIMRSKIQGDRPVSLVGFSLAARAIYVCLMILAERRQFGLIDSVVLMGTPAPSESRVWLTMKSVVSGRLVNVYSESDYLLGFLYRTSNIHFGLAGLQKIQGADGVENHNVSNLANGHLRYSLLVGKILKDIHWEDLDLNA

SEQ ID NO: 2 - protein, 377 aa, synthetic construct
  source 1..377: note = Amino acid sequence of FgTad2
MQSLQNRLTHNEDADKENQPPSASQLSESLARLELQDNKSILTSPKPVTPVIPPAPIFTDPAVVAERAMHMKFTEAALDMARLALQTNETPVGCVLVHNGRIIARGMNATNVTRNGTRHAEFMALGALLSYPPKDGPRTTYLKPKAENQSEAGSETSSIDSGPPDEGNEDGAKGHLYPYGQKCHPDARVDRSIIRESILYVTVEPCVMCASLLRQLGIKKVYFGAVNDKFGGTGGVFSIHANSLPVSADGQTASAHPTPKPAQLPDGSGTLGVSYPPGGGDGGNIESGYEIEGGWGRDEAVGLLRRFYVQENGRAPVPRKKEGRAARLAAMLEGEGNGEETPTPDITTPIPDADSIDLPASTETLKEPQPLADRTNV

SEQ ID NO: 3 - protein, 428 aa, synthetic construct
  source 1..428: note = Amino acid sequence of FgTad3
MSTELAPEVSLTPDSAEAALPAKPEHRGPSEIVPNLLIDDFKEKQIRRGVLIPLKTTQEVRQDHTITHAYITRAPTRQANDVITALRNMRPDDNSANPLPHLRRCSKPGDLPAHLKTQFMNDTAVGRQIHTAKSTWIYIIIGEVKDFTQEELTEVLSIIEGDEGELFIEKIPIPLLAPTSQVQAAMWSSQFWPTVYRKNNPLGPHPSIVARGTEDIKDDSSVWMALAHRVALQAKETGIGEAIGAVIVQRDGGKVELVGVAGDARWHQECGPLGGTSNPMTHCVVRAISMVAQKLVRHERRAAGLPFNPPNLDYDAFQDGPLLEIEKKCFVQEHPNKDGYLCHGLELYVTHEPCVSCSMGILHSRMGKAVFATHMPRSGGLSSDDRPDGGGRGLGLFWRRELNWSLMAWEWERDGVPNLPPLDPITHV

SEQ ID NO: 4 - DNA, 1959 bp, synthetic construct
  source 1..1959: mol_type = other DNA; note = Nucleotide sequence encoding the Ame1
atgacccgcggcgggcgaacaagttcggtccctcgcaggccagtcgacctgactagcaacctctctatcgctgagaaaaacgatctgacaactttgatcacagctattacagagaagcttcacaatgatatcagtatcatttttgattcacctccagtcacccctactctaggtgacttcagtgacattgagaatgtcgatatcggccatcaccactggctggctctgtcactccagacagatggcaaggagaacgtagacccaagacattccaatctaagcaagaatatccccaaaccagtaccaacagacaaaattacagatcagtcccgaggtagtgagggatccgaggccatcacacctcaactcagggagctaaagaaggaggctctgatattcttcagaaagtggcagtcaatctttctacagcgccttcgtgatatcagcgtcactgaaccaaacggctctcaacctaacatgcgtggccgtggtcgtggtcgtggaagtgcacgaggtgcaagaggtggccgtgtcgcacgtggtggaaggggagagggtcgtggaggtctgactttggctactggtccccccaagactccttccactcacatcgatcgcgagctgtctcgacggttcccccctatccccacctctctgtggactctaccactagagaagcgaaagcttctactacacatctcactcttgattgtcatttcactccaggagtatcttgcatatgcgcgaatgactctcctcaaccttacttcatctctcaatcttcctctgaaagtactacatgaggaagaaaagcgaatcgcacaaggactcagccaacttgcagtcgactcggtcatcgagcaggcttcacaacagagtcaggatgagaacaaaccagtattgaagtcaaaggcacgtatcgctgccatgactggacctttcagacttggaacgcagcttatcgccgtggggatcggaactgcccatggcggacacggcttgcgactcccaacagctgcagggctattggggcccatggctgataatggtccatctactggctcaatgctgggaatctacagtgcgagaccaacttgcaagatgattgagtcgttttcccgcgagatccaagattttggcctcatgcctcttcggtgtgaatcccaaggggactatatggatgcaacgtcaattccatctaccgctcgacgccttcgcttggtcattgctatcaacggttgggtaacggacaaagacaatctcacaggatcttggaaaatccttggtgataatgcggaagtgtatattctgcgatgggagatgaatgtgctttccaacataggcactgcccttgagacagttatcagaagctcagcttggactgcagcaaaacaggccatctcttcgcgaacaattttcaagaacctcatcgaagcaacttggccaatggaacttatgaaagtgagcaagatcatcgataacccctggagcatggggatggttcgcgccgagaaagccggtgctgttcttgcggacgcaatcatgcgcagcaagattcaaggtgaccgcccagtgtcccttgtcgggttcagcctagcagctcgagccatctatgtgtgtctgatgatacttgctgaacgtcgccagtttggtctcatcgattccgtggttttgatgggcacaccagctccttcagagagccgtgtctggttgactatgaagagtgtcgtatctggccgtctggtcaacgtctactctgagagcgactatctccttggattcttatatcgtacttccaacatccatttcgggctggcaggccttcaaaagatacagggcgcggatggtgtcgagaatcacaacgttagcaaccttgcgaacgggcacttgcgttattcactccttgttggaaagattctcaaggacattcattgggaggacctggaccttaacgcttag

SEQ ID NO: 5 - DNA, 1134 bp, synthetic construct
  source 1..1134: mol_type = other DNA; note = Nucleotide sequence encoding the FgTad2
atgcaatctctccagaatcgacttacccacaatgaggatgcagataaagaaaaccaaccccccagcgcatctcagctgtcggagtctcttgcccgcctggagcttcaagacaacaaatccatcttgacttctcccaagccagtcacgccagtcatcccacctgcaccgatattcacagaccctgctgtggtagctgagcgagcgatgcacatgaagtttacagaagcagctcttgatatggcccggctcgctcttcaaaccaacgaaacccctgttgggtgcgttcttgttcataatggacggattattgcacggggcatgaatgcgaccaatgtcactcgaaatggtactcgtcacgccgagttcatggcgctcggtgctttgctatcttaccccccaaaggatgggcctagaacgacttatctcaagcccaaagctgagaaccagtctgaagcaggttctgagacgtcttccattgattcgggacctccagatgaaggtaatgaagatggagccaaaggacacctctacccctacggacaaaagtgccaccctgatgcgcgagtcgacagatcgatcatcagagaaagcatcttgtacgtaacggttgagccatgtgtcatgtgtgcttctctattgcgtcagctcggcattaaaaaagtctacttcggcgctgtcaacgacaagtttggcggaactggcggtgttttcagcattcatgccaattcccttcccgtcagtgcggatggacagactgctagtgctcatcccaccccgaaacctgcacagctcccagacggcagtggtactttgggtgtctcatatcctccaggcggaggcgatggcggaaacattgaatctggctatgagatcgaaggtggctgggggcgcgatgaagccgttggcctactccgacgtttctatgtccaggagaacggacgagcgcctgtacctcgcaagaaggaagggcgcgcagcccgactagccgctatgctggaaggtgaaggtaatggagaagagaccccaacacctgatattacgacacctattccagatgccgattctatcgacctcccagcttcaacagaaaccctcaaggagccccagccgctggcagatcgcaccaatgtctag

SEQ ID NO: 6 - DNA, 1287 bp, synthetic construct
  source 1..1287: mol_type = other DNA; note = Nucleotide sequence encoding the FgTad3
atgtccaccgaactggctcctgaggtctccctcacccctgattcggccgaagcggctctgccagccaagcccgagcatcgcggcccctctgagattgtgccaaaccttttgattgatgactttaaggagaaacagatccggcgcggtgtcttgatacccctcaagacaacgcaagaggttcgacaagatcataccatcacccatgcatacatcacacgagcacctaccaggcaggccaacgatgtcatcacggccttgcgtaacatgcgaccagatgacaatagcgctaaccccctacctcatctgcgacgatgttctaagcctggtgatctgcctgcccaccttaaaacccagttcatgaatgacactgctgtgggccgccagattcatacagcgaagtcgacttggatttacatcatcatcggagaagtcaaggacttcacacaagaggagctcaccgaagttttatctatcatcgagggcgacgagggcgagctcttcattgaaaagatccccatccccctgcttgcgccaacatcacaagtacaagcagccatgtggtcttcacagttttggcccaccgtatatcggaagaacaaccccttaggaccacaccctagtatagtagcccggggcactgaagatatcaaggatgacagttctgtttggatggcactagcccatcgtgttgctttacaagctaaagaaaccgggatcggcgaagctatcggagctgtaatcgtccagagagatggtggaaaagtcgaactggtcggggttgcgggagacgctcgctggcaccaggagtgcggcccgttggggggtaccagcaaccctatgacacactgcgttgtccgtgctatcagcatggtggctcagaagcttgttcgacatgagagacgagctgctggcctcccttttaacccacccaatctcgattacgatgcattccaggacggaccactccttgagattgagaagaagtgctttgtacaggagcacccgaacaaggacggctatctctgtcatggccttgagctttatgtcactcatgagccctgcgtatcatgctctatgggcatactccattctcgaatgggcaaagctgtctttgccacgcacatgcctcgcagtggaggattgagctcagatgatcgtccggatgggggtggaagaggattgggactcttctggagacgagagctcaactggagcttgatggcttgggaatgggaacgtgacggcgtgcccaacttgcctcctcttgatcccatcactcatgtttag

SEQ ID NO: 7 - DNA, 1959 bp, synthetic construct
  source 1..1959: mol_type = other DNA; note = Nucleotide sequence of AME1 gene after codon optimization in E. coli
atgacccgcggcggccgcaccagcagcgtcccgcgccgtccggtggatctgaccagcaatctgagcattgcggaaaaaaacgatctgaccaccctgattaccgccattaccgaaaaactgcacaatgatatcagcattatttttgatagcccgccggtgaccccgaccctgggcgattttagtgacatcgaaaatgttgatattggccaccatcattggctggccctgagcctgcagaccgatggtaaagaaaatgtggatccgcgccatagtaacctgagcaaaaacattccgaaaccggtgccgaccgataaaatcaccgatcagtcacgcggcagcgaaggtagcgaagcgattaccccgcagttacgtgaactgaaaaaagaagccctgattttttttcgcaaatggcagagcattttcctgcagcgccttcgtgatatcagcgtcactgaaccaaacggctctcaacctaacatgcgtggccgtggtcgtggtcgtggaagtgcacgcggcgcccgcggcggtcgcgtggcgcgtggcggtcgtggcgaaggccgcggtggcctgaccctggcgaccggcccgccgaaaaccccgagcacacatattgatcgcgaactgagtcgccgtttcccgccgatcccgaccagcctgtggaccctgccgctggaaaaacgcaaactgctgctgcatatcagcctgctgattgtgattagcctgcaggaatacctggcgtacgcccgcatgaccctgctgaacctgaccagcagcctgaacctgccgctgaaagtgctgcacgaagaagaaaaacgcattgcgcagggcctgagccagctggcggtggattcggtgattgaacaggcaagccagcagagccaggacgaaaacaaaccggtgctgaaaagcaaagcgcgcattgcagcgatgaccggtccgtttcgtctgggtacccagctgattgccgtgggcattggcaccgcccatggtggccatggcttacgcctgccgaccgcggcaggcctgctgggcccgatggccgataacggtccgagcaccggcagcatgctgggcatttacagcgcgcgtccgacgtgcaaaatgatcgaaagctttagccgcgaaattcaggatttcggcctgatgccgctgcgctgtgaaagccagggcgattatatggatgccaccagcattccgagcacggcccgccgcctgcgcttggtgattgcgattaatggctgggtgaccgataaagataacctgaccggcagctggaaaattctgggtgataacgccgaagtgtacattctgcgctgggaaatgaatgtgctgtctaacattggcaccgcgctggaaaccgtgattcgcagctcagcgtggaccgcggccaaacaggcgattagcagccgtaccatttttaaaaatctgattgaagccacctggccgatggaactgatgaaagttagcaaaattattgataacccgtggagcatgggcatggtgcgcgccgaaaaagccggcgcggtgctggccgatgccattatgcgtagcaaaattcagggtgatcgcccggtgagcctggtaggctttagcctggcggcgcgcgcgatttatgtttgcctgatgattctggcggaacgtcgccagtttggcctgattgatagcgtggtgctgatgggaacgccggcgccgagcgaaagccgtgtgtggctgaccatgaaaagcgtggtgtcaggtcgcctggtgaacgtgtatagcgaatcggattatctgctgggcttcctgtatcgtaccagcaatattcattttggcctggcgggcctgcaaaaaattcaaggtgcggatggcgttgaaaaccacaacgttagcaatctggccaatggccatctgcgctacagcctgctggtgggcaaaattctgaaagatatccactgggaggatctggatctgaacgcgtaa

SEQ ID NO: 8 - DNA, 1134 bp, synthetic construct
  source 1..1134: mol_type = other DNA; note = Nucleotide sequence of FgTAD2 gene after codon optimization in E. coli
atgcagagcctgcagaaccgcctgacccataacgaagacgcggataaagaaaaccagccgccgagcgcgagccagctgagcgaatcgctggcacgtctggaactgcaggataataaaagcattctgaccagcccgaaaccggtgaccccggttattcctccggcaccgatctttaccgatccggccgtggtggcagaacgtgccatgcacatgaaatttaccgaagcagcgctggatatggcgcgcctggccctgcagaccaatgaaaccccggtgggttgcgtgctggtgcacaacggccgcatcattgcgcgtggtatgaacgccaccaacgtgacccgcaatggcacccgccatgcagaattcatggcgctgggtgcgttgctgagctacccgccgaaagatggcccgcgcaccacctatctgaaaccgaaagcggaaaaccagagcgaagcgggcagcgaaaccagcagcattgatagcggcccgccggatgaaggcaatgaagacggcgcgaaaggccacctgtatccgtatggccagaaatgccatccggatgcgcgcgtggatcgtagcattattcgtgaaagcattctgtatgtgaccgtggaaccgtgcgtgatgtgtgcgagcctgctgcgtcagctgggtattaagaaagtgtactttggcgcggtgaacgataaatttggcggtaccggcggcgtgtttagcattcatgcgaatagcctgccggtgagcgcggatggccagacggcgagcgcgcatccgaccccgaaaccggcccagctgccggatggctcaggcaccctgggtgttagctatccgccgggcggtggcgatggcggtaacattgaaagcggctatgaaattgaaggtggctggggtcgcgatgaagcggtgggcctgctgcgtcgcttctatgtccaggaaaacggccgcgcgccggtgccgcgcaaaaaagaaggccgcgcagcgcgtctggcggcgatgctggaaggcgaaggcaatggcgaagaaaccccgacgccggatatcaccacccctattccggatgcggatagcattgatctgccggcgagcaccgaaaccctgaaagaaccgcagccgctggcggatcgtaccaatgtgtaa

SEQ ID NO: 9 - DNA, 1287 bp, synthetic construct
  source 1..1287: mol_type = other DNA; note = Nucleotide sequence of FgTAD3 gene after codon optimization in E. coli
atgagcaccgaactggcgccggaagtaagcctgaccccggatagcgcggaagccgcgctgccggcgaaaccggaacatcgcggtccgagcgaaattgtgccgaatctgctgattgatgattttaaagaaaaacagattcgccgcggtgtgctgattccgctgaaaaccacccaagaagttcgccaggatcacaccattacccatgcctatatcacccgcgcgccgacccgccaggcgaacgatgtgattaccgccctgcgcaacatgcgtccggatgataactcagcaaacccgctgccgcatctgcgtcgctgtagcaaaccgggcgatctgccggcgcatctgaaaacccagtttatgaacgataccgcggtgggccgccagattcacaccgcgaaaagcacctggatttatattattatcggcgaagttaaagacttcacccaggaagaactgaccgaagtgctgagcattatcgaaggcgatgaaggcgaactgtttatcgaaaaaattccgattccgctgctggcgccgaccagccaagtgcaggcggcgatgtggagcagccagttttggccgaccgtgtatcgtaaaaacaatccgctgggcccgcacccgagcattgttgcccgtggcaccgaagatattaaagatgatagcagcgtgtggatggccctggcgcaccgtgtggccctgcaggccaaagaaaccggcattggcgaagcgattggcgcggtgattgtgcagcgcgatggcggcaaagtggaactggtgggcgtggcgggcgatgcccgttggcaccaggaatgcggtccgctgggcggcaccagtaatccgatgacccattgcgtggtgcgcgcgattagtatggtagcgcagaaactcgtgcgccacgaacgtcgtgcggcgggcctgccgtttaatccgccgaatctggattatgatgcctttcaggatggtccgctgctggaaattgaaaaaaaatgctttgtgcaggaacatccgaataaagacggctatctgtgccatggcctggaactgtatgttacgcatgaaccgtgtgttagctgcagcatgggcattctgcatagccgtatgggcaaggcggtgtttgccacccacatgccgcgcagcggcggcctgtcaagcgatgatcgcccggatggcggtggccgcggcctgggcctgttttggcgtcgtgaactgaattggagcctgatggcctgggaatgggagcgcgatggcgtcccgaacctgccgccgctggatccgattacccacgtgtaa

SEQ ID NO: 10 - DNA, 44 bp, synthetic construct
  source 1..44: mol_type = other DNA; note = Primer AME1-pYES2-F
tcactatagggaatattaagcttctacgcaaaaacggcatcatg

SEQ ID NO: 11 - DNA, 43 bp, synthetic construct
  source 1..43: mol_type = other DNA; note = Primer AME1-pYES2-R
ccctctagatgcatgctcgagctaagcgttaaggtccaggtcc

SEQ ID NO: 12 - DNA, 44 bp, synthetic construct
  source 1..44: mol_type = other DNA; note = Primer pYES2-FgTAD2-4M-F
tcactatagggaatattaagcatgcaatctctccagaatcgact

SEQ ID NO: 13 - DNA, 45 bp, synthetic construct
  source 1..45: mol_type = other DNA; note = Primer pYES2-FgTAD2-4M-R
ccctctagatgcatgctcgagctagacattggtgcgatctgccag

SEQ ID NO: 14 - DNA, 40 bp, synthetic construct
  source 1..40: mol_type = other DNA; note = Primer pYES2-FgTAD3-F
tcactatagggaatattaagcatgtccaccgaactggctc

SEQ ID NO: 15 - DNA, 47 bp, synthetic construct
  source 1..47: mol_type = other DNA; note = Primer pYES2-FgTAD3-R
ccctctagatgcatgctcgagctaaacatgagtgatgggatcaagag

SEQ ID NO: 16 - DNA, 49 bp, synthetic construct
  source 1..49: mol_type = other DNA; note = Primer pYES2-Leu2-F
gaattaattctcatctttgacagctaactgtgggaatactcaggtatcg

SEQ ID NO: 17 - DNA, 45 bp, synthetic construct
  source 1..45: mol_type = other DNA; note = Primer pYES2-Leu2-R
ataatgcctttagcggcttaactgtccggtcgaaattcccctacc

SEQ ID NO: 18 - DNA, 50 bp, synthetic construct
  source 1..50: mol_type = other DNA; note = Primer pYES2-TRP1-F
gaattaattctcatctttgacagctttgtactgagagtgcaccatagatc

SEQ ID NO: 19 - DNA, 47 bp, synthetic construct
  source 1..47: mol_type = other DNA; note = Primer pYES2-TRP1-R
ataatgcctttagcggcttaactgtcctgatgcggtattttctcctt

SEQ ID NO: 20 - DNA, 43 bp, synthetic construct
  source 1..43: mol_type = other DNA; note = Primer PRSFDuet-AME-MCS1-F
agcagccatcaccatcatcaccacatgacccgcggcggccgca

SEQ ID NO: 21 - DNA, 47 bp, synthetic construct
  source 1..47: mol_type = other DNA; note = Primer PRSFDuet-AME-MCS1-R
ttatgcggccgcaagcttgtcgacttagtggtggtggtggtggtgcg

SEQ ID NO: 22 - DNA, 45 bp, synthetic construct
  source 1..45: mol_type = other DNA; note = Primer PRSFDuet-4M-MCS2-F
ttaagtataagaaggagatatacatatgcagagcctgcagaaccg

SEQ ID NO: 23 - DNA, 53 bp, synthetic construct
  source 1..53: mol_type = other DNA; note = Primer PRSFDuet-4M-MCS2-R2
taccgacgtcagcgatcgcgtggccggccttagtggtggtggtggtggtgcac

SEQ ID NO: 24 - DNA, 57 bp, synthetic construct
  source 1..57: mol_type = other DNA; note = Primer PRSFDuet-T3-MCS2-F
ccggccacgcgatcgctgacgtcaaggagatatacatatgagcaccgaactggcgcc

SEQ ID NO: 25 - DNA, 50 bp, synthetic construct
  source 1..50: mol_type = other DNA; note = Primer PRSFDuet-T3-MCS2-R
caaatttcgcagcagcggtttctttttacacgtgggtaatcggatccagc

SEQ ID NO: 26 - DNA, 19 bp, synthetic construct
  source 1..19: mol_type = other DNA; note = Primer PRS-MCS1-F
ggatctcgacgctctccct

SEQ ID NO: 27 - DNA, 20 bp, synthetic construct
  source 1..20: mol_type = other DNA; note = Primer PRS-MCS1-R
gattatgcggccgtgtacaa

SEQ ID NO: 28 - DNA, 20 bp, synthetic construct
  source 1..20: mol_type = other DNA; note = Primer PRS-MCS2-F
ttgtacacggccgcataatc

SEQ ID NO: 29 - DNA, 19 bp, synthetic construct
  source 1..19: mol_type = other DNA; note = Primer PRS-MCS2-R
gctagttattgctcagcgg

SEQ ID NO: 30 - DNA, 51 bp, synthetic construct
  source 1..51: mol_type = other DNA; note = Primer pCMV-AME1-F
actaaagggaacaaaagctggagctccaccctacgcaaaaacggcatcatg

SEQ ID NO: 31 - DNA, 50 bp, synthetic construct
  source 1..50: mol_type = other DNA; note = Primer AME1-SV40-R
caggctgcgcaactgttgggaagggcgactaagcgttaaggtccaggtcc

SEQ ID NO: 32 - DNA, 19 bp, synthetic construct
  source 1..19: mol_type = other DNA; note = Primer SV40-F
tcgcccttcccaacagttg

SEQ ID NO: 33 - DNA, 32 bp, synthetic construct
  source 1..32: mol_type = other DNA; note = Primer Sv40-R
ttaagatacattgatgagtttggacaaaccac

SEQ ID NO: 34 - DNA, 59 bp, synthetic construct
  source 1..59: mol_type = other DNA; note = Primer proCMV-SV40-F
gtggtttgtccaaactcatcaatgtatcttaatcaattacggggtcattagttcatagc

SEQ ID NO: 35 - DNA, 20 bp, synthetic construct
  source 1..20: mol_type = other DNA; note = Primer proCMV-R
ggtggagctccagcttttgt

SEQ ID NO: 36 - DNA, 53 bp, synthetic construct
  source 1..53: mol_type = other DNA; note = Primer proCMV-4M-F
actaaagggaacaaaagctggagctccaccatgcaatctctccagaatcgact

SEQ ID NO: 37 - DNA, 50 bp, synthetic construct
  source 1..50: mol_type = other DNA; note = Primer 4M-SV40-R
tcaggctgcgcaactgttgggaagggcgactagacattggtgcgatctgc

SEQ ID NO: 38 - DNA, 49 bp, synthetic construct
  source 1..49: mol_type = other DNA; note = Primer proCMV-FgTAD3-F
actaaagggaacaaaagctggagctccaccatgtccaccgaactggctc

SEQ ID NO: 39 - DNA, 50 bp, synthetic construct
  source 1..50: mol_type = other DNA; note = Primer FgTAD3-SV40-R
ttctagactcgagagatctgtcgacgatatctcaactccggagctgacat

SEQ ID NO: 40 - DNA, 19 bp, synthetic construct
  source 1..19: mol_type = other DNA; note = Nucleotide sequence of T7 promoter
taatacgactcactatagg

SEQ ID NO: 41 - DNA, 25 bp, synthetic construct
  source 1..25: mol_type = other DNA; note = Nucleotide sequence of lac operator
ggaattgtgagcggataacaattcc

SEQ ID NO: 42 - DNA, 48 bp, synthetic construct
  source 1..48: mol_type = other DNA; note = Nucleotide sequence of T7 terminator
ctagcataaccccttggggcctctaaacgggtcttgaggggttttttg

SEQ ID NO: 43 - DNA, 508 bp, synthetic construct
  source 1..508: mol_type = other DNA; note = CMV enhancer and promoter sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 44 - DNA, 384 bp, synthetic construct
  source 1..384: mol_type = other DNA; note = SV40 polyA signal sequence
tcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaa